(19) United States
(12) Patent Application Publication
(10) Pub. No.: US 2024/0125726 A1
(43) Pub. Date: Apr. 18, 2024

(54) FLEXIBLE SENSOR DEVICE FOR MOISTURE DETECTION

(71) Applicant: Henkel AG & Co. KGaA, Düsseldorf (DE)

(72) Inventors: Jing Yang, Shanghai (CN); Xun Mo, Shanghai (CN); WeiFeng Tan, Shanghai (CN); Yao Song, Shanghai (CN)

(21) Appl. No.: 18/355,499

(22) Filed: Jul. 20, 2023

Related U.S. Application Data
(63) Continuation of application No. PCT/CN21/72856, filed on Jan. 20, 2021.

(51) Int. Cl. G01N 27/12; A61F 13/42; G01N 27/04

(52) U.S. Cl. CPC G01N 27/12; A61F 13/42; G01N 27/048; A61F 2013/424

(57) ABSTRACT

A flexible sensor device for moisture detection, a detecting method using said device, and applications of the device are disclosed.

### FIELD

The present application generally relates to a flexible sensor device for moisture detection, especially to a flexible sensor device capable of being conveniently and removably arranged on an irregular surface such as personal clothing.

### BACKGROUND

For current clothes that need to be worn close to a user's skin (take a diaper for example), it is necessary to build-in at least a part of a moisture measuring device in non-woven fabric of a diaper which is as an example of an incontinent protector. Therefore, the non-woven fabric has to be tailor-designed and manufactured so to be sewn into the finished diaper. However, the finished diaper can be used once only. That is, they are disposable. Furthermore, comparing with clothes everyday worn by a user, the finished diaper is poor in comfort and fit aspects. Therefore, it is required to develop a flexible sensor device for moisture detection capable of being, at any time, arranged on clothes everyday worn by the user such as common incontinent protectors and being conveniently replaced by a new one, so as to reduce the usage costs.

Moreover, take a diaper for example again. A conventional moisture measuring sensor device usually takes capacitance change of electrodes, contacting an objected to be tested, as the basis of measurement. This is mainly for the purpose of saving electric power. However, such a design may result in a risk of a false alarm. That is, an alarm may be created when the moisture does not reach a level to create the alarm, such that the user is disturbed.

### SUMMARY

In order to solve the above issues, the present application is aimed at proposing a novel flexible sensor device for moisture detection, such that it can be conveniently connected to an irregular surface to be tested (i.e. a surface with varying roughness), especially be releasably connected to a user's private underwear or an incontinent protector (for example a diaper). Moreover, the measurement results are not prone to the risk of the false alarm.

According to one aspect of the present application, a flexible sensor device for moisture detection is proposed, wherein the flexible sensor device comprises a flexible sensing part and a signal processing and transmitting part which is connectable to the flexible sensing part, wherein the flexible sensing part includes a non-conductive flexible substrate and two or more conductive traces attached on a side face of the flexible substrate, two conductive traces, which are not in contact with each other, are chosen from the two or more conductive traces to constitute a pairs of conductive traces, the side face of the flexible substrate which is provided with the conductive traces is a side face in direct contact with a surface to be detected, and the signal processing and transmitting part has a detection module for detecting the capacitance and/or resistance between the conductive traces of the pair of conductive traces so as to determine the moisture content of the surface to be detected.

Optionally, when the flexible sensor device operates, the capacitance between the conductive traces of the pair of conductive traces is first detected; only after a detected value of the capacitance exceeds a predetermined standard, the resistance between the conductive traces of the pair of conductive traces is detected.

Optionally, the moisture content of the surface to be detected is deemed to be out of limits only when both the detected values of the capacitance and resistance between the conductive traces of the pair of conductive traces are out of limits.

Optionally, only one pair of conductive traces is chosen from the conductive traces to detect the capacitance value and the resistance value at the same time.

Optionally, two pairs of conductive traces are chosen from the conductive traces, one of which pairs is configure to detect the capacitance value, and the other of which pairs is configured to detect the resistance value.

Optionally, each of the conductive traces is formed by conductive ink printed on the flexible substrate, or by a conductive wire arranged on the flexible substrate.

Optionally, when the signal processing and transmitting part is connected to the flexible sensing part, the detection module is electrically connected to the conductive traces.

Optionally, a non-dry adhesive material is applied on the side face of the flexible substrate in direct contact with the surface to be detected; or alternatively hook & loop is provided between the side face of the flexible substrate provided with the conductive traces and the surface to be detected.

Optionally, a detection zone is defined in the side face of the flexible substrate provided with the conductive traces, wherein the conductive traces extend in the detection zone as long as possible.

Optionally, the conductive traces of the pair are parallel to each other, and extend in the detection zone to meander back and forth.

Optionally, the conductive traces of the pair meander back and forth in such a way that they are substantially parallel or perpendicular to a lengthwise direction of the detection zone.

Optionally, the signal processing and transmitting part is releasably connected to the flexible sensing part.

Optionally, two connector sheets are securely provided on the flexible substrate and are connected to free ends of the conductive traces of the pair respectively; wherein two conductive pieces are provided on the signal processing and transmitting part to be connected to the detection module, and when the signal processing and transmitting part is connected to the flexible sensing part, the connector sheets are in contact with the conductive pieces respectively.

Optionally, the signal processing and transmitting part also comprises a wireless transmission module so as to output data detected by the detection module and/or to send an alarm that the moisture content of the surface to be detected has been out of limits outwards.

Optionally, the signal processing and transmitting part comprise a closable and openable housing so as to selectively clamp the flexible substrate, and the detection module and the wireless transmission module are disposed in the housing.

Optionally, the housing comprises a first housing half part and a second housing half part which are hinged to each other, and the flexible substrate is able to be clamped between the first housing half part and the second housing half part.

Optionally, the flexible substrate is in the form of a film made of a flexible waterproof and electrically insulating material.

According to another aspect of the present application, a method for detecting the moisture content of a surface by the already mentioned flexible sensor device for moisture detection is proposed, wherein the method comprises:
contacting a flexible sensing part of the flexible sensor device with the surface to be detected in such a way that two or more conductive traces of the flexible sensing part in contactless with each other are in contact with the surface to be detected;
detecting the capacitance between a pair of conductive traces chosen from the two or more conductive traces first;
after detection of the capacitance arrives at a predetermined first condition, detecting the resistance between the pair of conductive traces or detecting the resistance between another pair of conductive traces chosen from the two or more conductive traces and different than the pair of conductive traces; and
after detection of the resistance arrives at a predetermined second condition, determining the current moisture content status of the surface to be detected on the basis of both the detected values of the capacitance and the resistance.

Optionally, the predetermined first condition comprises carrying out at least two detections of the capacitance, and each detection result is greater than a predefined capacitance value.

Optionally, the predetermined second condition comprises carrying out at least two detections of the resistance, and each detection result is less than a predefined resistance value.

According to another aspect of the present application, use of the already mentioned flexible sensor device for moisture detection on a diaper to form a diaper whose moisture content can be detected is proposed, wherein a side face of a flexible substrate of the flexible sensor device on which a conductive trace is provided is attached to non-woven fabric of the diaper.

Using the above mentioned technical measures of the present application, it is easy and low-cost to change a private hygienic absorbent product whose moisture content cannot be detected into a private hygienic absorbent product whose moisture content can be detected. Here, the private hygienic absorbent product includes but is not limited to a diaper, an Easy-Up diaper, sanitary towel or any other product which requires moisture monitoring. Therefore, it reduces the costs for the user who uses similar products. In the meanwhile, as the sensor device is replaceable, it can be more flexibly used. Besides, the flexible sensor device according to the present application can be readily arranged on any irregular surface to monitor its moisture content, such that the accuracy of moisture detection can be increased and the rate of false alarm can be reduced.

### BRIEF DESCRIPTION OF THE DRAWINGS

The principles and other aspects of the present application can be well understood by the following description in combination of the attached drawings. It should be noted that although the drawings may be given in different proportions for illustrative purpose only, they cannot be deemed to affect understanding to the present application. In the drawings:

FIG. 1 is a system chart schematically illustrating a flexible sensor device according to an embodiment of the present application, wherein the flexible sensor device is in data communication with a data processing device;

FIG. 2 is a top view schematically illustrating a flexible sensing part of the flexible sensor device according to the embodiment of the present application;

FIG. 3 is a lateral cross-sectional view schematically illustrating the flexible sensing part of the flexible sensor device of FIG. 1;

FIG. 4 is a view schematically illustrating the flexible sensing part is connected to a signal processing and transmitting part of the flexible sensor device according to the embodiment of the present application;

FIG. 5A is a perspective view schematically illustrating an example of the signal processing and transmitting part;

FIG. 5B is a diagram schematically illustrating the signal processing and transmitting part of the flexible sensor device according to the embodiment of the present application;

FIG. 5C is a view schematically illustrating that the flexible sensor device according to the embodiment of the present application carries out moisture detection; and

FIG. 6 is a flow chart schematically illustrating an example of a method for carrying out the measurement by the flexible sensor device of the present application.

### DETAILED DESCRIPTION OF EMBODIMENTS

In the drawings of the present application, features having the same configuration or similar functions are represented by the same reference numerals respectively.

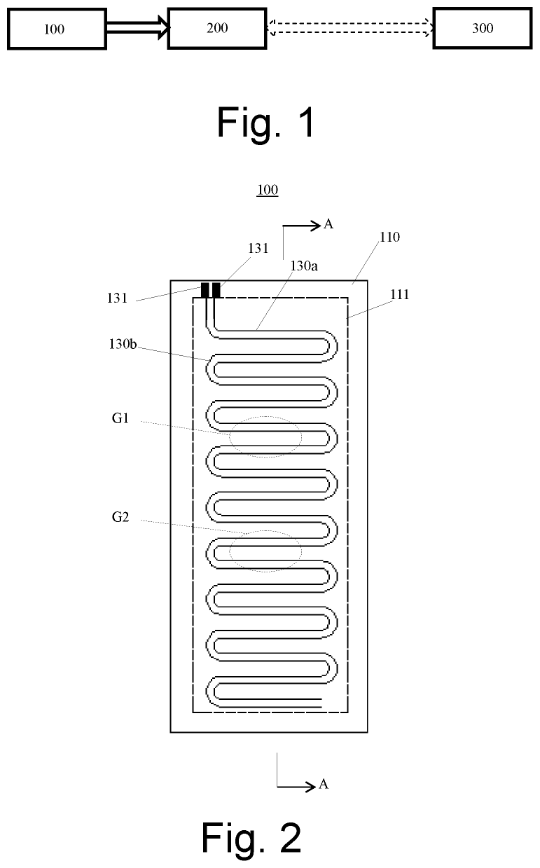

FIG. 1 is a system chart schematically illustrating a flexible sensor device according to an embodiment of the present application. The flexible sensor device described in the present application is applicable to private hygienic absorbent product which is able to be held against a user's skin. For instance, in the following description, the flexible sensor device according to the present application is mainly explained to be held against the user's common diaper such that they together act as a baby or adult diaper. However, it should be understood by one ordinary person in the art that the flexible sensor device according to the present application is alternatively applicable to detection of human skin moisture or can be used in any situation where moisture needs to be detected conveniently. Furthermore, the private hygienic absorbent product to which the technical solutions of the present application are applied includes but is not limited to diapers, Easy-Up diapers, and sanitary towels. Moreover, the term “common diaper” in the description shall be understood as a diaper lacking of a function of moisture content monitoring.

As shown by FIG. 1, according to the embodiment of the present application, the flexible sensor device generally comprises a flexible sensing part 100 and a signal processing and transmitting part 200. The flexible sensing part 100 is configured to be coupled to a user's common diaper which can be held against the user's skin, such that the diaper's moisture content is detectable. The signal processing and transmitting part 200 is able to process and control an electrical signal detected by the flexible sensing part 100 and/or transmit the received electrical signal and the processed data outwards, for example in a wired or wireless manner, such that the signal or data can be received by a data processing device 300.

The flexible sensing part 100 is in data communication with the signal processing and transmitting part 200 such that the electrical signal detected by the flexible sensing part 100 can be sent to the signal processing and transmitting part 200. FIG. 2 further illustrates an example of the flexible sensing part 100 according to the present application. As shown, the flexible sensing part 100 comprises a flexible substrate 110 and a pair of electrode traces attached on a side face of the flexible substrate 110. For instance, according to the present application's embodiment, the flexible substrate 110 is in the form of a film made of a flexible waterproof and electrically insulating material. Here, the flexible waterproof and electrically insulating material can be thermoplastic polyurethane (TPU). The pair of electrode traces are comprised of a pair of conductive traces 130a, 130b which are attached on the exposed side face of the flexible substrate 110 in such a way that the two traces are parallel to each other and do not intersect with each other. As shown by FIG. 1, the conductive traces 130a, 130b extends in a major projected area of the flexible sensing part 110 in a meandering manner. The flexible substrate 110 is shown as being substantially rectangular or elongated such that it is able to fit to an outer surface of the diaper. It should be understood by the ordinary person in the art that depending on actual requirements, the flexible substrate 110 can be formed as other shapes.

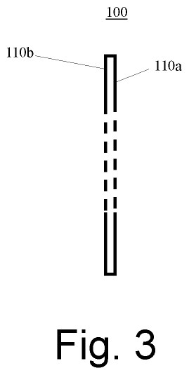

Further as shown by FIG. 3, the flexible substrate is enlarged and illustrated in a lateral cross-sectional view taken along a direction A-A of FIG. 2. As shown, the flexible substrate 110 comprises a first side face 110a and an opposing second side face 110b. In use, the first side face 110a is a side face away from the user's diaper, and the second side face 110b is a side face towards the user's diaper. The conductive traces 130a, 130b are secured on the second side face 110b. In this way, as the second side face 110b is in contact with the user's diaper, the conductive traces 130a, 130b will naturally contact the non-woven fabric of the user's diaper. In this case, as the flexible substrate 110 is made of the flexible waterproof and electrically insulating material, a portion of a circuit for sensing of capacitance and/or resistance can be formed between the non-woven fabric of the diaper contacted by the conductive traces 130a, 130b and the conductive traces 130a, 130b. Therefore, the capacitance and/or resistance of the non-woven fabric of the diaper in contact between the two conductive traces 130a, 130b can be monitored respectively so as to determine the moisture content of the corresponding non-woven fabric of the diaper.

For instance, a detection zone 111 is defined in the flexible substrate 110, especially on the second side face 110b of the flexible substrate 110. The detection zone 111 for example extends along a lengthwise direction of the flexible substrate 110 with a distance being left from either side edge of the flexible substrate. The two conductive traces 130a, 130b extends in the detection zone 111 in such a way that the two traces meander back and forth so as to make them as long as possible. For instance, each conductive trace can first extend along a first direction B1, substantially perpendicular to the lengthwise direction of the flexible substrate 110, from a border of the detection zone 111 to an another opposite border, and then extends along a second direction B2, exactly opposing the first direction B1, to the first mentioned border, and then extends along the first direction B1 again. As such, the trace extends in such a way that it meanders back and forth. Such meandering back and forth can ensure that each conductive trace is as long as possible in the detection zone 111 such that an area to be detected can be occupied as large as possible. Therefore, it can cover a region, possibly being wetted by moisture, as great as possible, so as to increase the accuracy of detection.

Furthermore, it should be understood by the ordinary person in the art that besides being substantially perpendicular to the lengthwise direction of the flexible substrate 110, the direction B1, B2 can follow or go against the lengthwise direction of the flexible substrate 110 or extend at an acute or obtuse angle relative to the lengthwise direction. Moreover, it should be understood by the ordinary person in the art that in the context of the present application “meandering back and forth” refers to that the targeted feature (for example the conductive trace) extends forwardly and inversely, not only linearly but also curvedly back and forth. Furthermore, although in the illustrated embodiment the conductive traces 130a, 130b extend parallel to each other, it should be understood by the ordinary person in the art that in an alternative embodiment the conductive traces can extend in such a manner that they are not parallel to each other.

According to an embodiment of the present application, the conductive traces 130a, 130b can be formed by printing conductive ink onto the flexible substrate 110 or alternatively by bonding a conductive metal wire like a copper wire onto the flexible substrate 110. A non-dry adhesive material can be applied to the second side face 110b of the flexible substrate 110 where the conductive traces 130a, 130b locate, such that the side face can be readily bonded to the diaper's surface to guarantee that the conductive traces 130a, 130b are in contact with the diaper's surface. For instance, a plurality of non-dry adhesive sections can be separately provided on the surface of the second side face 110b of the flexible substrate 110 or alternatively the non-dry adhesive material can be provided on the entire surface of the second side face. Prior to leaving the factory, a protective film can be arranged on the second side face 110b to cover the non-dry adhesive material and the conductive traces 130a, 130b. If required, the protective film can be torn off directly so as to bond the flexible substrate 110 onto the diaper's surface in such a way that the second side face 110b faces towards the diaper's surface. In an alternative embodiment, hook & loop can be provided between the second side face 110b and the diaper's surface so as to ensure that they can be releasably attached to each other. For instance, the non-dry adhesive material or the hook & loop can be located in one or more blank areas defined by sections of the conductive traces 130a, 130b perpendicular to the lengthwise direction of the flexible substrate 110 on the second side face 110b. Therefore, in order to increase the bonding strength between the second side face 110b and the diaper's surface, the sections of the conductive traces 130a, 130b can be unevenly spaced from each other along the lengthwise direction of the flexible substrate 110.

As shown by FIG. 2, two connector sheets 131 are securely provided on the flexible substrate 110 such that they are conductively connected to free ends of the conductive traces 130a, 130b respectively. The connector sheets 131 are for example made of a conductive material like metal. The two connector sheets 131 are separated from each other and secured on the flexible substrate 110, especially its second side face 110b, in a manner similar to the conductive traces. The two connector sheets 131 are adjacent to an edge of the flexible substrate 110 such that the signal processing and transmitting part 200 if clamping the flexible substrate 110 can be in contact with the connector sheets 131.

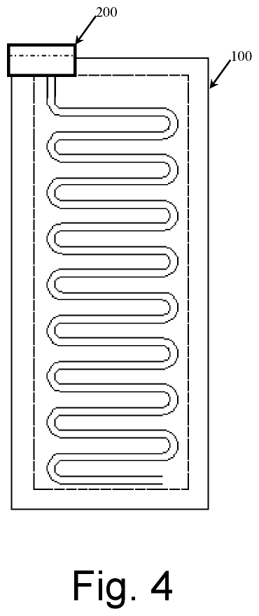

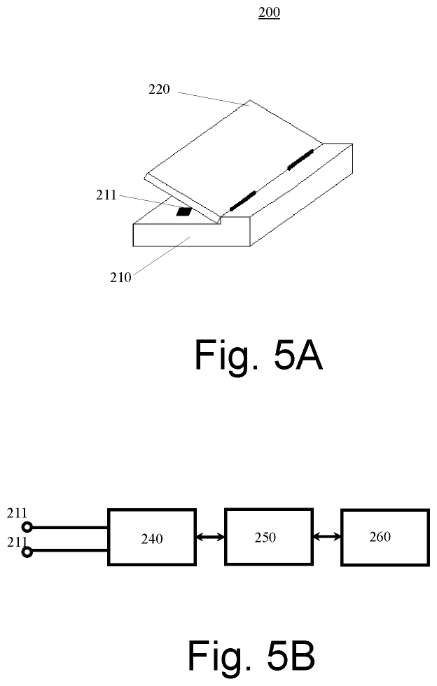

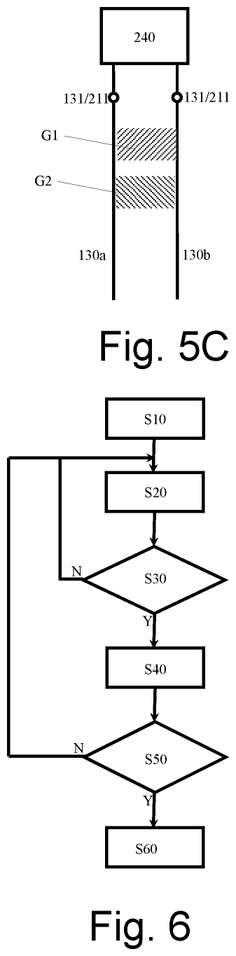

As shown by FIGS. 4 and 5A, the signal processing and transmitting part 200 includes a housing. The housing includes a first housing half part 210 and a second housing half part 220 which are hinged to each other. Received in the housing of the signal processing and transmitting part 200, especially the first housing half part 210 are several signal processing components such as an analog-to-digital convertor device, a control board, a wireless transmission module and so on. The first housing half part 210 and the second housing half part 220 can be hinged to each other by a pivotal shaft. In the meanwhile, a spring can be wound around the pivotal shaft to apply a force between the first housing half part 210 and the second housing half part 220 to let them separate. Furthermore, attractive magnets can be provided on surfaces of the first and second housing half parts 210 and 220 facing towards each other, respectively, such that an attractive force applied by the magnets can enable the first housing half part 210 to be close relative to the second housing half part 220, and thus the flexible substrate 110 to be clamped between the housing half parts. It should be understood by the ordinary person in the art that besides the magnetically attractive force, any other suitable measure such as a Snap-On mechanism can be used to ensure the closing between the two housing half parts. Furthermore, in the context of the present application, the housing half part is just a non-limiting/exemplary expression, and does not means that the volume or weight of the half part is definitely equal to or substantially equal to half of the volume or weight of the entire housing. Two conductive pieces 211 (only one of which is illustrated in FIG. 5A) are provided on a surface of the housing of the signal processing and transmitting part 200, especially on a surface of the first housing half part 210 and are located corresponding to the connector sheets 131, such that when the flexible substrate 110 is clamped between the first housing half part 210 and the second housing half part 220, the two conductive pieces 211 are in contact with the respective connector sheets 131.

Further as shown by FIG. 5B, for example, received in the housing of the signal processing and transmitting part 200 are a detection module 240, a data processing module 250 as well as a wireless transmission module 260. Besides, a power supply such as a rechargeable battery can be received in the housing to provide electric power to those modules for their operation. The detection module 240 is electrically connected to the two conductive sheets 211. A corresponding detection circuit is provided in the detection module 240 for sensing the capacitance and/or resistance of the circuit part connected between the two conductive sheets 211. It should be understood by the ordinary person in the art that any integrated circuit or dedicated chip available in the market can be used as the detection module 240 here. The detection module 240 can output an electrical signal which can be processed by the data processing module 250 connected thereto. For instance, the data processing module 250 can be in the form of a Micro-Computer (MCU) chip well known by the ordinary person in the art. Furthermore, the data processing module 250 is able to transmit the received electrical signal or the processed electrical signal to the data processing module 300 via the wireless transmission module 260, or alternatively the data processing module 250 is able to receive instructions from the data processing module 300 via the wireless transmission module 260. For instance, the wireless transmission module 260 includes a BLUETOOTH module, a WIFI module, an infrared data signal transmission module, a 5G signal transmission module or any other suitable wireless data transmission module. Correspondingly, the data processing module 300 can be a mobile-phone of the guardian, a personal computer or even a cloud server.

According to the embodiments of the present application, when the flexible sensor device is used, the flexible sensing part 100 of the flexible sensor device can be directly bonded to the non-woven fabric material of the user's diaper and at the same time the signal processing and transmitting part 200 can be clamped onto the flexible sensing part 100 such that the connector sheets 131 are in contact with the conductive pieces 211 respectively. Then, if one or more wetting regions such as wetting regions Q1 and Q2 caused by moisture existing in the non-woven fabric material and for example illustrated by FIG. 1, the part between the conductive traces 130a, 130b and the wetting regions Q1 and Q2 of the cloth material forms a portion of a signal sensing circuit, as shown by FIG. 5C, because of the direct contact of the conductive traces 130a, 130b of the flexible sensing part 100 with the cloth material. For instance, the detection module 240 can be used to detect the capacitance and/or resistance of the portion of the signal sensing circuit, as required. As areas of the wetting regions Q1 and Q2 occupied between the conductive traces Q1 and Q2 will be varied, this must result in variation of the capacitance and/or resistance to be detected. Therefore, by continually monitoring such variation, one will be able to determine whether the cloth material is over-wetted. If the moisture content exceeds a predefined value and an alarmed need be raised, the alarm can be sent by the data processing module 250 to the mobile-phone of the guardian via the wireless transmission module 260, so as to notify the guardian that the diaper shall be replaced by a new one. Therefore, in order to monitor whether the wetting region(s) exists in the detection zone 111 of the flexible substrate 110 and how much the moisture content of the wetting region(s) is as accurately as possible, each conductive trace shall be as long as possible in the detection zone 111, as already mentioned, so as to occupy an area as large as possible.

Using the flexible sensor device of the present application as already mentioned, it is not necessary to purposely build-in the conductive traces, as detection electrodes, into clothing fabrics in advance. Therefore, it makes easier to manufacture clothes such as diapers. Furthermore, the user experience can be improved because the flexible sensor device can be directly attached to his/her private cloth material so as to detect the cloth material's moisture content. Besides, it is easy to replace the flexible sensing part 100 of the flexible sensor device so as to reduce the usage costs.

In a conventional moisture content detecting method, the capacitance is usually detected, i.e. continually monitoring the capacitance of a portion of a circuit to be detected is used to determine whether the moisture content is out of limits. However, this method of detection of the capacitance is disadvantageous in that the detecting results are too sensitive, and thus sometime although the moisture content variation exists (even if this variation may be caused by excessive environmental humidity), the moisture content of the cloth material itself does not arrive at the extent to which the alarm shall be sounded. In this case, the alarm will be false.

In order to improve the reliability of the detecting result, FIG. 6 schematically illustrates a flow chart of an example of a method for carrying out detection by the flexible sensor device according to the present application. It should be understood by the ordinary person in the art that steps of the method described here can be coded as a program which is stored and can be executed by the data processing module 250 or the data processing device 300 if desired.

First, at step S10, the flexible sensing part 100 is attached over a surface to be detected, and self-inspection is carried out after the signal processing and transmitting part 200 is connected to the flexible sensing part 100 (i.e. the former is clamped onto the latter such that the connector sheets 131 are in contact with the conductive pieces 211 respectively). For instance, the detection module 240 can be instructed by the data processing module 250 to carry out trial detection of data, so as to make sure whether an abnormal statue such as short circuit exists; and/or the wireless transmission module 260 can be instructed to carry out self-inspection, so as to make sure whether any malfunction exists.

Then, at step S20, the detection module 240 is instructed to operate, so as to detect the capacitance between the two conductive pieces 211. In the meanwhile, the detected data can be supplied to the data processing module 250. At step S30, it is judged whether the detected capacitance value is out of limits according to predetermined criterion. Here, the predetermined criterion may be capacitance detection criterion that is well known and followed by the ordinary person in the art. For instance, an empirical value can be defined in advance for capacitance detection. If an actually detected capacitance value exceeds the empirical value, the current capacitance value between the two conductive pieces 211 is deemed to be out of limits.

If the judgment result is “NO (N)” at step S30, the process returns to step S20 and the detection module 240 continues to be instructed to operate so as to detect the capacitance between the two conductive pieces 211 and supply the detected data to the data processing module 250. If the judgment result is “YES (Y)” at step S30, the process goes to step S40. At step S40, the detection module 240 is instructed to operate so as to detect the resistance between the two conductive pieces 211 and supply the detected data to the data processing module 250. Optionally, the judgment result at step S30 can be made dependent on several capacitance detections. That is, if the capacitance value of a first detection is out of limits, the detection module 240 is instructed to continually operate so as to detect the capacitance between the two conductive pieces 211 and judge whether the capacitance value of a second detection is out of limits. Only when the capacitance values of several successive detections (for example at least two detections) are out of limits, the judgment result can be “YES” at step S30.

Then, at step S50, it is judged whether the detected resistance value is out of limits according to predetermined criterion. Here, the predetermined criterion may be resistance detection criterion that is well known and followed by the ordinary person in the art. For instance, an empirical value can be defined in advance for resistance detection. If an actually detected resistance value is less than the empirical value, the current resistance value between the two conductive pieces 211 is deemed to be out of limits.

If the judgment result is “NO” at step S50, the process returns to step S20 and the detection module 240 continues to be instructed to operate so as to detect the capacitance between the two conductive pieces 211 and supply the detected data to the data processing module 250. If the judgment result is “YES” at step S50, the process goes to step S60. At step S60, the wireless transmission module 260 is instructed to transmit an alarm signal to the mobile-phone of the guardian to warn him/her that the diaper shall be checked or replaced. In an alternative embodiment, provided in the housing of the signal processing and transmitting part 200 can be an alarm device such as a beeper, such that an alarm is sounded by the beeper.

Optionally, the judgment result at step S50 can be made dependent on several resistance detections. That is, if the resistance value of a first detection is out of limits, the detection module 240 is instructed to continually operate so as to detect the resistance between the two conductive pieces 211 and judge whether the resistance value of a second detection is out of limits. Only when the resistance values of several successive detections (for example at least two detections) are out of limits, the judgment result can be “YES” at step S50.

The above mentioned method according to the present application can ensure that a false alarm can be avoided such that the reliability of the detecting results for moisture content can be improved. Moreover, as the resistance detection is carried out only in case that the capacitance value is detected to be out of limits, the accuracy of detection can be improved with satisfying requirements of saving electric power under the premise that the flexible sensor device is able to operate in a prescribed period.

It should be understood by the ordinary person in the art that the flexible sensor device according to the present application is not limited to be applicable to the field of diapers only. For instance, the flexible sensing part 100 of the flexible sensor device can be designed to be directly attached onto the human skin (for example, his/her face), so as to detect the moisture content of the human skin's surface, or alternatively the flexible sensing par can be directly attached onto any other irregular surface, so as to detect the moisture content of the surface.

Furthermore, although in the already explained embodiments two conductive traces 130a, 130b are described to monitor the capacitance value and the resistance value, it should be understood by the ordinary person in the art that in an alternative embodiment more than two (i.e. two or more) conductive traces which are in not contact with each other can be provided, such that a pair of conductive traces can be chosen from these conductive traces as the pair of conductive traces 130a, 130b in the already explained embodiments. As an alternative, it is also possible that one pair of conductive traces is chosen from these conductive traces to carry out detection of the capacitance separately, and another pair of conductive traces is chose from the conductive traces to carry out detection of the resistance value separately.

Although some specific embodiments of the present application have been described here, they are given for illustrative purposes only and cannot be thought to constrain the scope of the present application. Furthermore, it should be understood by the skilled person in the art that the embodiments described in the specification can be arbitrarily combined with each other. Without departing from the spirit and scope of the present application, various alternations, replacements and modifications can be thought out.

1. A flexible sensor device for moisture detection, characterized in that the flexible sensor device comprises a flexible sensing part (100) and a signal processing and transmitting part (200) which is connectable to the flexible sensing part (100), wherein the flexible sensing part (100) includes a non-conductive flexible substrate (110) and two or more conductive traces attached on a side face of the non-conductive flexible substrate (110), wherein two conductive traces (130a, 130b) not in contact with each other are chosen from the two or more conductive traces as a pair of conductive traces, the side face of the non-conductive flexible substrate (110) which is provided with the conductive traces is a side face in direct contact with a surface to be detected, and the signal processing and transmitting part (200) has a detection module (240) for detecting a capacitance and a resistance between the conductive traces (130a, 130b) of the pair of conductive traces so as to determine the moisture content of the surface to be detected.

2. The flexible sensor device as recited in claim 1, characterized in that when the flexible sensor device operates, the capacitance between the conductive traces (130a, 130b) of the pair of conductive traces is first detected only after a detected value of the capacitance exceeds a predetermined standard, the resistance between the conductive traces (130a, 130b) of the pair of conductive traces is detected.

3. The flexible sensor device as recited in claim 2, characterized in that the moisture content of the surface to be detected is deemed to be out of limits only when both the detected values of the capacitance and the resistance between the conductive traces (130a, 130b) of the pair of conductive traces are out of limits.

4. The flexible sensor device as recited in claim 1, characterized in that only one pair of conductive traces is chosen from the two or more conductive traces to detect the capacitance value and the resistance value at the same time.

5. The flexible sensor device as recited in claim 1, characterized in that two pairs of conductive traces is chosen from the two or more conductive traces, one pair of which is configure to detect the capacitance value and the other pair of which is configured to detect the resistance value.

6. The flexible sensor device as recited in claim 1, characterized in that each of the conductive traces (130a, 130b) is formed by a conductive ink printed on the flexible substrate (110) or by a conductive wire arranged on the non-conductive flexible substrate (110).

7. The flexible sensor device as recited in claim 1, characterized in that when the signal processing and transmitting part (200) is connected to the flexible sensing part (100), the detection module (240) is electrically connected to the conductive traces (130a, 130b).

8. The flexible sensor device as recited in claim 1, characterized in that a non-dry adhesive material is applied on the side face of the non-conductive flexible substrate (110) in direct contact with the surface to be detected.

9. The flexible sensor device as recited in claim 8, characterized in that hook & loop is provided between the side face of the non-conductive flexible substrate (110) provided with the conductive traces and the surface to be detected.

10. The flexible sensor device as recited in claim 9, characterized in that a detection zone (111) is defined in the side face of the non-conductive flexible substrate (110) provided with the conductive traces (130a, 130b), wherein the conductive traces (130a, 130b) extend in the detection zone (111) as long as possible.

11. The flexible sensor device as recited in claim 10, characterized in that the conductive traces (130a, 130b) of the pair are parallel to each other.

12. The flexible sensor device as recited in claim 11, characterized in that the conductive traces (130a, 130b) of the pair extend in the detection zone (111) to meander back and forth.

13. The flexible sensor device as recited in claim 12, characterized in that the conductive traces (130a, 130b) meander back and forth in such a way that they are substantially parallel or perpendicular to a lengthwise direction of the detection zone (111).

14. The flexible sensor device as recited in claim 1, characterized in that the signal processing and transmitting part is releasably connected to the flexible sensing part (100).

15. The flexible sensor device as recited in claim 5, characterized in that two connector sheets (131) are securely provided on the non-conductive flexible substrate (110) and are connected to free ends of the conductive traces (130a, 130b) of the pair respectively; wherein two conductive pieces (211) are provided on the signal processing and transmitting part (200) to be connected to the detection module (240), and when the signal processing and transmitting part (200) is connected to the flexible sensing part (100), the connector sheets (131) are in contact with the conductive pieces (211) respectively.

16. The flexible sensor device as recited in claim 1, characterized in that the signal processing and transmitting part (200) also comprises a wireless transmission module (260) so as to output data detected by the detection module (240) and/or to send an alarm that the moisture content of the surface to be detected has been out of limits outwards.

17. The flexible sensor device as recited in claim 16, characterized in that the signal processing and transmitting part (200) comprise a closable and openable housing so as to selectively clamp the non-conductive flexible substrate (110), and the detection module (240) and the wireless transmission module (260) are disposed in the closeable and openable housing.

18. The flexible sensor device as recited in claim 17, characterized in that the closeable and openable housing comprises a first closeable and openable housing half part (210) and a second closeable and openable housing half part (220) which are hinged to each other, and the flexible substrate (110) is able to be clamped between the first closeable and openable housing half part (210) and the second closeable and openable housing half part (220).

19. The flexible sensor device as recited in claim 1, characterized in that the flexible substrate (110) is in the form of a film made of a flexible waterproof and electrically insulating material.

20. A method for detecting the moisture content of a surface to be detected by a flexible sensor device for moisture detection, characterized in that the method comprises:
contacting a flexible sensing part (100) of the flexible sensor device with the surface to be detected in such a way that two or more conductive traces of the flexible sensing part (100) in contactless with each other are in contact with the surface to be detected;
detecting a capacitance between a pair of conductive traces (130a, 130b) chosen from the two or more conductive traces first;
after detection of the capacitance arrives at a predetermined first condition, detecting resistance between the pair of conductive traces (130a, 130b) or detecting the resistance between another pair of conductive traces chosen from the two or more conductive traces and different than the pair of conductive traces; and
after detection of the resistance arrives at a predetermined second condition, determining a current moisture content status of the surface to be detected on the basis of both the detected values of the capacitance and the resistance.

21. The method as recited in claim 20, characterized in that the predetermined first condition comprises carrying out at least two detections of the capacitance, and each detection result is greater than a predefined capacitance value.

22. The method as recited in claim 21, characterized in that the predetermined second condition comprises carrying out at least two detections of the resistance, and each detection result is greater than a predefined resistance value.

23. The of a flexible sensor device for moisture detection as recited in claim 1 which is a diaper, wherein the side face of the non-conductive flexible substrate (110) of the flexible sensor device on which a conductive trace (130a, 130b) is provided is attached to a non-woven fabric material of the diaper.
